(19) United States
(12) Patent Application Publication
(10) Pub. No.: US 2020/0253790 A1
(43) Pub. Date: Aug. 13, 2020

(54) DIAPER NOTIFICATION SYSTEM

(71) Applicant: Zanthion Inc., San Francisco, CA (US)

(72) Inventor: Philip Regenie, San Francisco, CA (US)

(73) Assignee: Zanthion Inc.

(21) Appl. No.: 16/789,023

(22) Filed: Feb. 12, 2020

Related U.S. Application Data
(60) Provisional application No. 62/804,562, filed on Feb. 12, 2019.
(60) Provisional application No. 62/812,307, filed on Mar. 1, 2019.

(51) Int. Cl. A61F 13/42

(52) U.S. Cl. CPC A61F 13/42; A61F 2013/424

(57) ABSTRACT

A system is presented for detecting the presence and contents of a soiled diaper and creating a notification once a soiled diaper is detected and assessed. The system incorporates mechanisms to detect the quantity of diaper contents, the type of diaper contents and to store and communicate relevant data.

### CROSS REFERENCE TO RELATED APPLICATIONS

This application claims priority to U.S. Provisional Patent Application 62/804,562, filed on Feb. 12, 2019, which is hereby incorporated by reference. This application further claims priority to U.S. Provisional Patent Application 62/812,307, filed on Mar. 1, 2019, which is hereby incorporated by reference.

### FIELD OF THE INVENTION

The present invention relates generally to diaper systems, and more specifically, to a diaper notification system with a flexible circuit incorporated into a diaper that allows for determination of diaper soiling and further provides for data collection.

### BACKGROUND OF THE INVENTION

Diapers today have use for children and adults alike in association with purposes to allow a wearer to urinate or defecate without the use of a toilet. In typical uses, diapers absorb or contain waste products to prevent soiling of the outer clothing or the external environment. Diapers are typically worn by babies and young children, people with disabilities and seniors. When diapers become wet or soiled, they require changing, generally by a second person such as a parent or caregiver. Failure to change a diaper on a sufficiently regular basis can result in sepsis, urinary tract infections and skin problems around the area of the diaper.

A number of more specific adverse results are associated with diaper use. For example, skin irritation can occur due to trapped heat and moisture. Skin barrier damage may result from chafing or rubbing. The ammonia in trapped urine and the enzymes present in stool may cause inflammation and damaged skin tissue. Many diapers contain dyes, perfumes and other materials that pose a risk of allergic reaction. Usage of diapers and related products such as padded underwear and pads may result in fungal and bacterial infections, typically Candida albicans and Staphylococcus aureus, respectively. Diaper use may also cause flare-ups of chronic skin conditions, such as psoriasis and eczema.

Each of such adverse results are exacerbated by the untimely changing of a diaper, often due to concealment of the presence of soiling due to embarrassment on the part of the wearer, or general unawareness of soiling on the part of the third party tasked with changing the diaper. As a result, the changing of the soiled diaper does not occur in time to prevent such adverse results. Often, especially in the cases of small children and persons with disabilities, the wearer lacks the capability to ask for help in changing the diaper. As such, persons wearing diapers often spend an excessive period of time in soiled diapers in a manner that negatively impacts their health.

A specific instance where a lack of resources to change soiled diapers persists is a senior community. Senior communities often have very few staff for large numbers of residents. The process of “rounding,” where personnel start at a specific point and travel room to room during scheduled intervals, remains the most common method of administering care for changes in diapers, showers, and other needs among senior community residents. Similar mechanisms exist in day care facilities for infants, small children and persons with disabilities. Due to the time that passes between such intervals, diaper users often suffer the need to wear soiled diapers for excessive periods of time. Among senior communities especially, skin damage and urinary tract infections remain pervasive and a common cause of complaint in community care centers. As such, unmet needs remain related to the challenges associated with diaper soiling in such contexts.

A variety of solutions have previously existed to attempt to address such unmet needs. However, such solutions generally feature inadequacies that have not optimally addressed the unmet meets. For instance, a variety of technologies feature embedded sensors within traditionally known diaper constructs. However, embedded sensors often require disposal, and the configuration of such a system is generally burdensomely expensive. Moreover, manufacturing processes traditionally associated with the creation of diapers involve the utilization of rolls of material, which do not lend themselves well to embedding sensors. Relatedly, wires and connectors associated with sensors pose burdensome expense and complexity.

Additionally, the complexities associated with the incorporation of electronic components into a diaper pose great difficulties. For instance, placing a power input near the sensor poses challenges associated with interference in sensing or interfere with the charging process. Such a process may further may cause danger for the wearer. Batteries can be expensive, hazardous and add heft and weight to a diaper construct. Moreover, external power supplies generally lack ergonomic design and often do not last for a period of several months before replacement.

A variety of challenges associated with data collection and management additionally present in association with existing technologies. For instance, the hardware associated with the sensors in a diaper sensing technology often lacks the capability to collect and organize data in a fashion effectively and efficiently presentable to a user or a third party. A host of data security concerns also accompany attempted solutions due to the sensitive nature of the data collected. Existing systems generally lack the capability to efficiently collect data related to who responded to a soiled diaper, the time of response, and the time of changed diaper. Existing systems also lack customization to aggregate, store, analyze and transmit data relevant to the diapers to parties who may assist in the process of addressing soiled diapers. Finally, existing systems lack the ability to integrate data related to soil diapers with complementary technologies, such as electronic health records, senior care management systems, child care management systems, and other systems related to more general health of the users of systems related to soiled diaper management.

The following prior art is known to Applicants:

U.S. Pat. No. 6,580,013 describes a diaper wetness monitoring system. This system includes an elongated compliant sensor installable within the front portion of the diaper and having an inner conductive layer, a hydrophilic porous middle layer containing a water-soluble ionizable salt, and an outer conductive layer. When urine contacts the sensor, the salt dissolves to form a highly conductive volume of solution that causes an electrical short circuit between inner and outer layers. The short circuit enables a low voltage direct current to activate an alarm mechanism. However, such a system poses inconveniences associated with the installation and use of the sensor within commonly accepted diaper systems. Moreover, such system lacks the capability to effectively collect, organize, store and collect data for use by third party systems and caregivers. The system utilizes components that are burdensomely expensive to utilize, particularly when disposable in nature.

Chinese Patent CN 203953960U describes an environmentally friendly diaper with a urine sensing function. The environmentally friendly diaper comprises a diaper body, an inner pad and a sensing device. However, the components (particularly the wires) associated with such configuration are expensive, and potentially dangerous to the wearer in the described configurations.

U.S. Pat. No. 5,264,830 describes an apparatus for sensing a wet diaper. The apparatus includes a sensing means for sensing wet conditions in the diaper. The apparatus further incorporates a signal means connected to the sensing means for producing a signal substantially concurrently with sensing the wet conditions in the diaper. The configurations described, however, pose a risk of discomfort and danger to the wearer. Moreover, the apparatus as a whole adds burdensome heft and weight in comparison to a traditional diaper. This item also lacks an ergonomic charging mechanism. Moreover, such system lacks the capability to effectively collect, organize, store and collect data for use by third party systems and caregivers.

U.S. Pat. No. 5,570,082 describes a remote wetness sensor for diapers. The device consists of a combination of an antenna, a nonlinear element and two electrodes. Upon a wet condition the resistance between the two electrodes decrease, resulting in an increase in coupling between the antenna and the nonlinear element. The ensuing nonlinear interaction between the antenna and the background field gives rise to harmonics of the field which are detected by a receiver, which triggers an alarm. A problem associated with such system is its integration within the lining of a diaper. This integration is expensive and difficult to accomplish.

The elements of this system in its described configuration pose potential danger to the user. Moreover, such system lacks the capability to effectively collect, organize, store and collect data for use by third party systems and caregivers.

United States Patent Application Publication 20120206265 describes a disposable diaper with a wetness alarm system. The system described consists of a disposable diaper structure; b) a disposable diaper telltale conductive strip; c) an attachable, removable, reusable battery-powered sensor-transmitter device; and d) a wireless receiver with an alarm signal. While this system describes a particular mechanism for identifying each diaper, in its current configuration it is burdensomely expensive. Moreover, such system lacks the capability to effectively collect, organize, store and collect data for use by third party systems and caregivers. Further, such a system lacks an ergonomic charging mechanism and adds burdensome heft and weight in comparison to a traditional diaper. Additionally, such system lacks the ability to integrate data related to soiled diapers with complementary technologies, such as electronic health records, senior care management systems, child care management systems, and other systems related to more general health of the users of systems related to soiled diaper management. Another specific problem associated to this disclosed art is that it lacks any sort of mitigation mechanism associated with the inadvertent disposal or misplacement of the reusable unit, which is a particular problem due to its usage in association with disposable diapers.

As such, a need has developed for an improved soiled diaper and notification system.

### SUMMARY OF THE INVENTION

The present invention relates to a novel and nonobvious system of detecting the presence and contents of a soiled diaper and creating a notification once a soiled diaper is detected and assessed. The system features a detection module. In one aspect, the detection module comprises an accelerometer used in association with determining the mass of the contents of an attached diaper. In another aspect, the detection module comprises a plurality of electrodes for use in association with determining the conductivity of the contents of an attached diaper, which provides an indication of the type of contents. An aspect of the system is a communicative linkage which allows for the external transfer of messages to gateways and clients to provide notifications associated with diaper soiling events.

### BRIEF DESCRIPTION OF THE FIGURES

FIG. 1 presents a top down view of the diaper comprising a conductive strip and an attached detection module.

FIG. 2 presents a cross-sectional side view of the diaper demonstrating the placement of an attached detection module in accordance with intended usage.

FIG. 3 presents a cross-sectional side view of the detection module demonstrating an exemplary placement of its components in an embodiment of the invention.

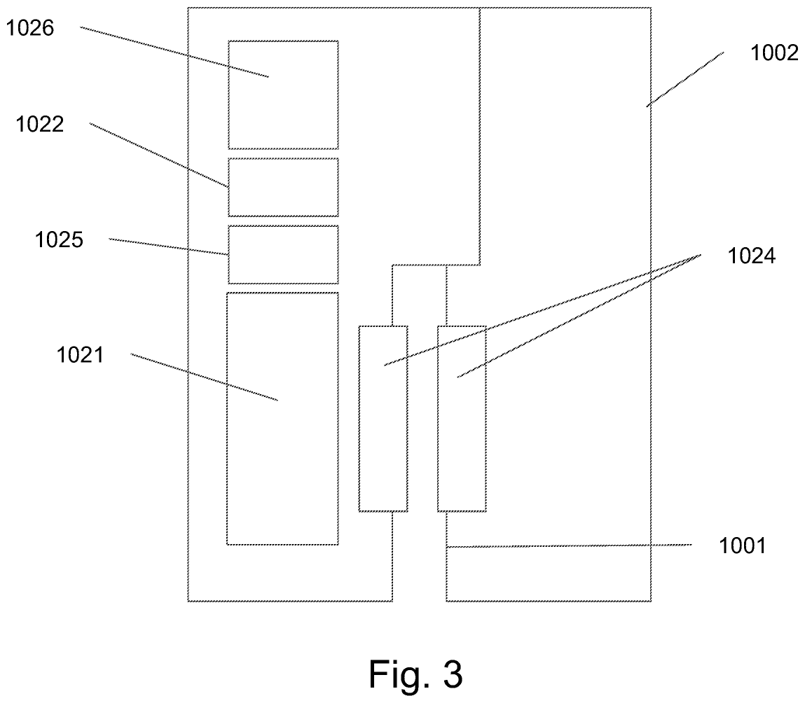

### DETAILED DESCRIPTION

The present invention relates to a novel and nonobvious system of detecting the presence and contents of a soiled diaper and creating a notification once a soiled diaper is detected and assessed.

In the preferred embodiment of the invention, a detection module 1000 is presented. The detection module comprises an inside surface 1001 and an outside surface 1002. Generally speaking, the inside surface 1001 is the surface that is intended for placement between a diaper 5000 and the wearer's body. Also generally speaking, the outside surface 1002 faces away from the wearer's body and is intended for placement during use external to the diaper 5000 and not between the diaper 5000 and the wearer's body.

In the preferred method of use, the detection module 1000 is configured for placement on a diaper 5000 edge 5001. To accomplish such placement, the detection module 1000 further comprises an inside member 1010 comprising a portion of the inside surface 1001 forms a flat, narrow shape. The configuration of the inside member 1010 reflects intent to provide for the wearer when placed between a diaper 5000 and the wearer's body.

The detection module 1000 further comprises a main body 1020. In the preferred embodiment, the main body 1020 is linked to the inside member 1010 at a fulcrum point, optionally by a pivot 1050. In such configuration, the inside member 1010 rotates around the pivot 1050 to bring it closer to or further away from the main body 1020. In an embodiment, the detection module 1000 further comprises a locking mechanism to hold the inside member 1010 in place relative to the main body 1020. During an intended use, the inside member 1010 and the main body 1020 when placed in close proximity and locked in place by the locking mechanism can hold a diaper 5000 edge 5001 between the inside member 1010 and main body 1020 for secure attachment to a diaper 5000 during the intended use. In an embodiment, the inner surfaces of the inside member 1010 and the main body 1020 employ ridging or “alligator grip” as recognized by those skilled in the art to further allow a secure grip onto a diaper 5000. In an embodiment, the locking mechanism utilizes elastic tension to create pressure and thereby lock the detection module 1000 in place. In an alternative embodiment, the locking mechanism comprises a spring clamp placed at the fulcrum point configured to place outward force onto the inside member 1010.

In the preferred embodiment, the main body 1020 further comprises a battery 1021. In the preferred embodiment the battery 1021 is rechargeable in accord with mechanisms well known in the art. The battery 1021 optionally consists of a lithium-ion battery or lithium polymer battery. In an embodiment, the battery 1021 consists of a 3.7 volt rechargeable battery. In the preferred embodiment, the battery 1021 comprises dimensions corresponding to a housing of slightly larger dimensions to accommodate the batter 1021, said housing placed within and attached to the main body 1020. In the preferred embodiment, the battery 1021 comprises the dimensions of 21 millimeters by 19 millimeters by 2 millimeters. In an embodiment, the housing comprises ridges to create a snug fit to hold the battery 1021 in place within the housing. In an embodiment, the battery 1021 features a protective plastic seal and an o-ring to insulate the battery 1021 and prevent contact with any user.

In the preferred embodiment the main body 1020 further comprises an accelerometer 1022. The accelerometer 1022 is configured to provide a measurement in the dynamics of the diaper 5000 during movement to determine the mass change of the diaper 5000. One skilled in the art understands that a diaper 5000 has a specific mass. The accelerometer 1022 during intended use functions to assist in determining the change in the mass of the diaper 5000 and its contents. One skilled in the art will appreciate that when mass increases the acceleration of the vertical movement of the diaper 5000, the accelerometer 1022 can function in accordance with Newton's second law equation, which teaches that the acceleration of an object is inversely proportional to the mass of an object, to reveal the relationship of the acceleration of the diaper 5000 and the mass of the diaper 5000 and its contents. Generally speaking, as the mass of the diaper 5000 increases, the acceleration of the diaper 5000 will decrease. In an aspect of the invention, the accelerometer 1022 is useful in determining the mass of the diaper 5000 and its contents, and thus the diaper 5000's soiled or clean state, by measuring the acceleration of the diaper 5000.

In the preferred embodiment, the invention further comprises a plurality of electrodes 1024. In an aspect of the invention, the plurality of electrodes 1024 is connected to an integrated circuit in communication with the contents of the connected diaper 5000. The plurality of electrodes 1024 facilitates a measurement in the change of current travelling through the contents of the diaper 5000 to assess the level of soiling. As one skilled in the art will appreciate, when the main body 1020 featuring a plurality of electrodes 1024 is attached to the diaper 5000, a completed circuit having a specific resistance in accordance with Ohm's law is created. Differing substances contained within a diaper 5000, for instance feces, urine and tomato juice, each feature different resistances and conductivity. It is an aspect of the invention that a measurement of conductivity or the variance in conductivity, optionally determined through utilization of the plurality of electrodes 1024, optionally one or more direct wire connectors and the battery 1021, allows for a determination of which different substance is contained within a diaper 5000. In another aspect of the invention, when used in conjunction with the accelerometer 1022 to determine the quantity of a substance within a diaper 5000, in its preferred embodiment, the invention can determine both the amount and type of substance contained within a diaper 5000.

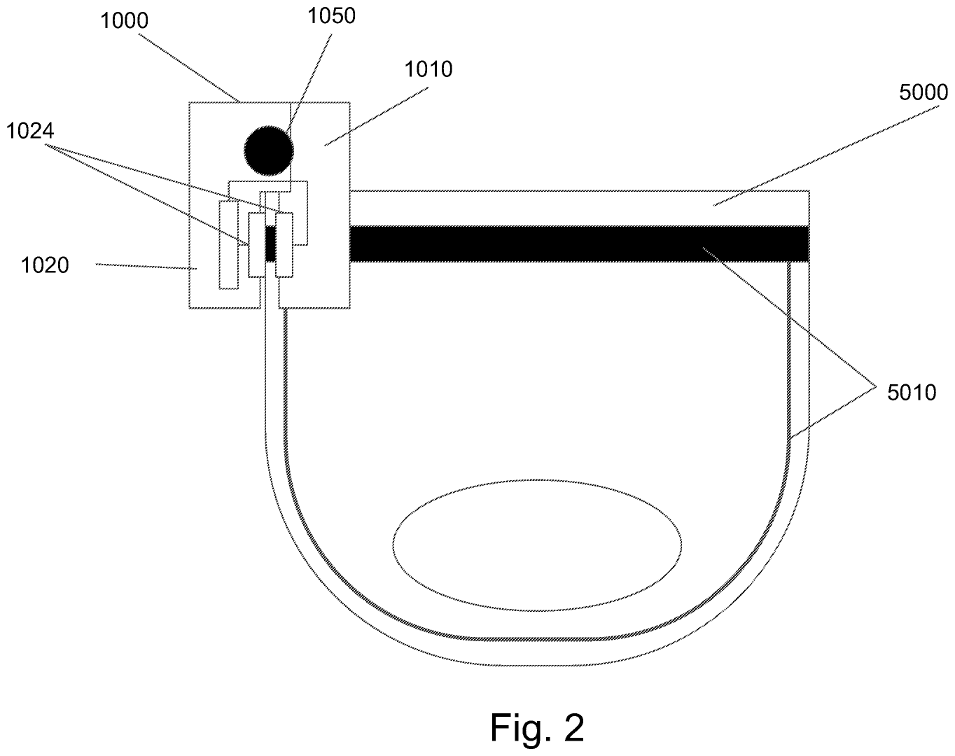

It is an aspect of the invention to have a diaper 5000 that comprises a conductive interior. In the preferred embodiment, the conductive interior is created by the presence of dry substrate placed within the diaper 5000 to facilitate conductivity. In an embodiment, the conductive interior is created by the presence of a conductive strip 5010, optionally in conjunction with the presence of dry substrate placed within the diaper 5000. The present inventors have recognized that a solution, optionally a solution of diluted tomato juice, when sprayed onto the interior surface of the diaper 5000 and then dried to become the dry substrate facilitates conductivity through the diaper 5000 to complete the circuit through the diaper, optionally in contact with the conductive strip 5010 and/or the plurality of electrodes 1024. The dry substrate in the preferred embodiment is located within the diaper 5000 such that it is in direct contact with the conductive strip 5010 to allow for the completion of a circuit when a detection module 1000 is attached to the diaper 5000. In the preferred embodiment, the dry substrate comprises tomato juice, optionally dissolved with water. The present inventors have recognized that a dry substrate comprised of dried diluted tomato juice both facilitates conductivity through the diaper 5000 and also is unlikely to cause adverse health interaction due to contact of the dry substrate with the skin. The conductivity of dried diluted tomato juice as the dry substrate also has the favorable advantage of being measurably different than the conductivity of urine and the conductivity of feces, which materially benefits the intended uses of embodiments of the invention. In the preferred embodiment, therefore, the diaper 5000 comprises both a dry substrate and a conductive strip 5010 configured such that during intended use a circuit may be completed including both the diaper 5000 and the detection module 1000 by placing the plurality of electrodes 1024 in contact with the conductive strip 5010 as depicted in FIG. 2. In an alternative embodiment, the dry substrate may completely replace the conductive strip 5010.

The present inventors have recognized the ranges of electrical conductivity of substances particularly relevant to the invention during intended uses. The International System of Units unit for conductivity is Siemens per meter (S/m), often linked directly to the total suspended solids (TSS) of a substance. A measurement relevant and assistive to determine the type of contents within a diaper 5000 during the intended use of the invention is Degrees Brix, which reflects the sugar content of an aqueous solution, particularly helpful in temperature ranges relevant to the intended use of the invention. For example:

The electrical conductivity of tomato juice ranges from 0.4 to 2.65 S/m as the TSS increases from 4.0 to 13.5 Degrees Brix in the temperature range of 25 to 80 degrees C. Similarly, in case of orange juice the electrical conductivity varied from 0.3 to 1.56 S/m as the TSS increased from 11.5 to 24 Degrees Brix.

Separately, urine electrical conductivity ranges from 1.49 to 16.85 mS cm−1. One skilled in the art will appreciate that urine conductivity has a positive relation with osmolality. During one intended use of the invention, the inventors have determined that an aspect may be particularly useful for performing electrical conductivity tests in different types of stone patients.

In another potentially relevant case, the conductivity for feces may vary between approximately 1 and 50 mS/cm. The present inventors have determined during an intended use of the invention that when the substance between the plurality of electrodes 1024 is substantially feces, the electrical conductivity may be between the conductivity for air and urine. In various aspects, the conductivity measurements for tomato juice, urine, feces and other substances are useful in determining the contents contained within a diaper 5000.

An embodiment of the invention features at least one direct wire connector to assist in the completion of the integrated circuit. In an exemplary configuration, the battery 1021 is connected to the integrated circuit by one or more direct wire connectors linked to a plurality of electrodes 1024. In an aspect of the invention, at least one of the plurality of electrodes 1024 is placed on the opposite exterior face of the inside member 1010 from at least one other of the plurality of electrodes 1024. During an intended use of the invention, in a configuration whereby at least one of the plurality of electrodes 1024 is on the inside of the inside member 1010 and at least one of the plurality of electrodes 1024 is on the outside of the inside member 1010, the circuit is completed by a conductive strip 5010 placed within the diaper 5000 which is in turn connected to the spray that is electrically conductive in the center of the diaper 5000. The material in the center of the diaper 5000 determines the resistance level, which can be measured. The measurement of the resistance level and/or conductivity then indicates what material is in the center of the diaper 5000.

In the preferred embodiment the main body 1020 further comprises a memory unit 1025. In the preferred embodiment of the invention, the memory unit 1025 is utilized for “store and forward” purposes as will be appreciated by one skilled in the art, and maintains data even when there is no external connection. Then, when external connection is reestablished, data contained within the memory unit 1025 can be communicated. The memory unit 1025 is configured to store information relating to the rate of change of electrical connectivity detected by the plurality of the electrodes 1024 and information relating to acceleration detected by the accelerometer 1022. In the preferred embodiment of the invention, the memory unit 1025 will have a capacity from within the range of 2 megabytes to 8 megabytes, inclusive, roughly equivalent to 500,000 data entry points.

In various embodiments of the invention, the memory unit 1025 is a memory unit supplied by one of a variety of well-known vendors of an integrated circuit to a specification well known in the art. In an exemplary embodiment, the memory unit 1025 is compatible with and configured for use with generally known standards for BLE standards for memory byte array communication.

In the preferred embodiment the main body 1020 further comprises a communicative linkage, such as a Bluetooth low energy chip 1026. The Bluetooth low energy chip 1026 is configured to wirelessly communicatively connect with external computing devices to transfer information sensed by the plurality of electrodes 1024 and the accelerometer 1022 and/or stored on the memory unit 1025. In one example, the Bluetooth low energy chip 1026 is configured to communicatively connect with the Zanthion SMART Hub, described in U.S. Provisional Patent Application 62/812,307, filed on Mar. 1, 2019, which is hereby incorporated by reference in its entirety.

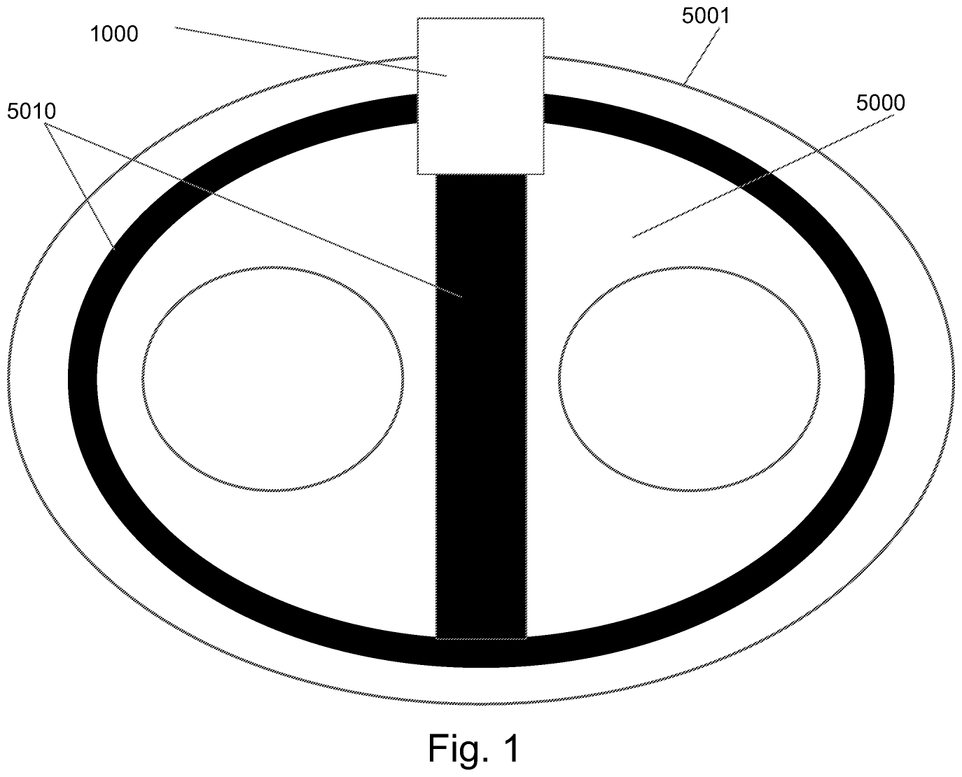

In another aspect of the invention, a diaper 5000 is presented. In the preferred embodiment, the diaper 5000 comprises a conductive strip 5010. The conductive strip 5010 in the preferred embodiment continuously traverses from the anterior aspect of the interior of the diaper 5000 to the posterior aspect of the interior of the diaper 5000 and to the inferior aspect of the interior of the diaper 5000, as demonstrated in FIG. 1. The conductive strip 5010 is configured to detect the level of moisture present contained within the interior of the diaper 5000. In an exemplary configuration, the conductive strip 5010 consists of a well-known readily available magnetic tape. In an alternative exemplary configuration, the conductive strip 5010 consists of a well-known readily available wire. The wire could consist of a cloth wire as well known in the art. In an exemplary aspect of the invention, the conductive strip 5010 completes a circuit further comprising a plurality of electrodes 1024, the battery 1021, the contents of the diaper 5000, a voltage measurement circuit and the integrated circuit. In the preferred embodiment, the voltage measurement circuit is configured to detect the resistance of the completed circuit in comparison with the ranges of resistance relating to the relevant ranges of various substances contained within the diaper in association with Ohm's law in accordance with mechanisms and methods as well understood by those skilled in the art. In the preferred embodiment, the integrated circuit is configured to store the resistance values in the circuit that exists inside the diaper 5000 and acceleration values as collected by the accelerometer 1022 and then transmit such values to a gateway in accordance with mechanisms and methods as well understood by those skilled in the art.

In an embodiment, the detection module 1000 optionally comprises a ribbed lever. The ribbed lever, in its preferred configuration, features notches to enhance grip in order to facilitate easy removal of the detection module 1000 from a diaper 5000. The ribbed lever functions by increasing the lever arm to improve on the mechanical advantage. In an embodiment, the ribbed lever comprises a non-stick surface. The present inventors have recognized that the ribbed lever facilitates easier usage of embodiments of the invention by persons with low grip strength or persons with arthritis.

The external surface of the detection module 1000 in the preferred embodiment consists of a sealed enclosure featuring a water-resistant surface. In an embodiment, the water-resistant surface consists of ABS or acetal plastic. The present inventors have recognized that such materials have favorable qualities in association with extrusion, avoidance of sunlight related delay, and avoidance of skin reactivity.

In the preferred embodiment, the invention is useful within the context of a broader system via a communicative linkage. In addition to optionally being communicatively connected to the data management system described in U.S. Provisional Patent Application 62/812,307 already incorporated by reference herein, the detection module 1000 in such embodiment is intended to communicatively link with phones, tablets, and television gateways in proximity to the detection module 1000. It is intended that configurations of the invention as described herein enable communication with any other data management system operating on any “smart device” enabled with Bluetooth, wifi or cellular capability in accordance with mechanisms well known in the art.

In one example, the detection module 1000 communicates with a gateway in accord with a Bluetooth low energy handshake in accordance with the BLE 4.0 specification streaming a byte array with the raw data. The gateway in turn interprets the raw data to determine if a change of diaper 5000 or soiling event has occurred based on a completed circuit or a resistance change. Following the occurrence of a detected soiling event, the gateway publishes the event to a message queue using a java implemented library in association with methods well understood by those skilled in the art. A client subscribed to the message queue receives the message following the message's addition to the queue in accordance with methods well understood by those in the art. During an intended use, the client recognizes the type and severity of the message and matches it up against the originator to determine the relevant person, time, and event. In an embodiment, the present invention is configured to interact with the gateway and client further described within U.S. Provisional Patent Application 62/812,307; such that the gateway collects, analyzes and communicates event data to the client. Optionally, the gateway and client are further linked to backend functionality configured to aggregate the collected data and predict future events in accordance with methods well understood by those skilled in the art.

Upon connecting with a gateway, in its preferred embodiment the invention is configured to communicate information of a defined categorical set, determinable in association with the presence or absence of one of the following states as will be appreciated by one skilled in the art: 1) electrodes contacting each other and voltage—no diaper 5000 battery charged, 2) electrodes touching each other no signal—battery not charged, 3) standard resistance level of diaper 5000—battery charged and clip in place, 4) Urine resistance level—battery charged urine present, 5) Feces resistance level—battery charged feces present, 6) some other resistance level—battery charged unknown substance. The communicated information, optionally via a Bluetooth low energy chip 1026, is formatted and transmitted in accord with mechanisms that are well understood by those skilled in the art. Specific categories of information delivered to the gateway optionally include:

a. “Connected/Disconnected” generally signifies whether the detection module 1000 is connected to a particular diaper 5000. This information is detectable within the context of a plurality of electrodes 1024 connected to the conductive strip 5010 to complete the circuit.
b. “Battery Level” is a readout of the percentage of battery life remaining. In configurations of the invention utilizing BLE standards, battery state readout is sent by default accompanying each message, optionally in addition to one or more additional message components, for instance an identifier, a latitude/longitude location, altitude, sensor values and/or timestamp.
c. “Heartbeat” is a standard type of information recognizable to those skilled in the art to assist in the determination of whether a device is connected. In general, a heartbeat refers a repeating communication delivered at a regular interval regardless if the intended activity occurs. In an exemplary configuration of the invention, a heartbeat is delivered at a one minute interval. If two minutes pass without the detection of a heartbeat in such configuration, the device is considered disconnected from the network. Additional information may be delivered in association with the heartbeat message, including, for example, the battery status.
d. “Soiled/Clean” is an information category signifying whether an attached diaper 5000 has experienced a soiling event. In an exemplary configuration, one of three states relating to “soiled/clean” category may be communicated via a connected gateway: 1) urine, 2) feces, and 3) dry.
e. “Location” provides a location based on last known location provided by sensors associated with a gateway. In an example of the invention, the determination location takes place in accord with to the following exemplary use cases:
1) in the case where the connected gateway is a connected smartphone device, the location for that device with the last known GPS location; or
2) in the case where the gateway is stationary associated with a particular location, the particular location is provided.
   f. The location information typically consists of a set of latitudinal and longitudinal coordinates.

When connected with an external system via a gateway, it is intended that the information collected by the detection module 1000 and its components may be stored in accordance with the processes associated with external systems. In various embodiments, such information is stored in the memory unit 1025 prior to transfer via the communicative linkage.

In the preferred embodiment, each detection module 1000 features its own unique address. In one such embodiment, the unique address consists of a MAC address. In an alternative embodiment, the unique address is generated via a hashing algorithm based on the detection module's 1000 individual MAC address and a unique database internal identifier for the detection module 1000 in accordance with methods well known in the art.

In various embodiments of the invention, the communications generated by its components are subject to security mechanisms. In an embodiment, TLS and HTTPS standards are utilized to safeguard communications in accord with mechanisms well known in the art. In a various embodiments of the invention, login credentialing systems as well understood in the art are utilized with the relevant user interfaces associated with the invention.

In an embodiment of the invention, the gateway comprises a mobile device such as a smartphone. In alternative embodiments, the gateway comprises a fixed site wireless gateway, such as a commercial-grade router. In an exemplary embodiment, the invention may be configured to communicate with a plurality of gateways depending on utilization circumstances.

The following provides an example of an unencrypted message depicting a set of relevant information, as will be easily interpreted by one skilled in the art, generated during the intended usage of the invention:

{

“appId”: 1329,
“gateway”: {

“battery”: 100,
“id”: 10512,
“location”: {

“altitude”: 0,
“latitude”: 0,
“longitude”: 0,
“name”: “”

},
“type”: “14”,
“userId”: 2875,
“uuid”: “cx_396F_ibQ”,
“version”: 890

},
“id”: 34,
“seqId”: “1580821721257”,
“source”: {

“battery”: 100,
“id”: 10513,
“location”: {

“altitude”: −11,
“latitude”: 38.46494674682617,
“longitude”: −77.38789367675781,
“name”: “”

},
“type”: “−1”,
“userId”: 2875,
“uuid”: “cx_396F_ibQ”,
“version”: −1

},
“timestamp”: 1580821721257,
“value”: [

38.464947,
−77.38789,
−11

]

}

The present inventors have recognized a particular advantage of the embodiments and configurations described above in that they require a substantially lower manufacturing cost, especially in comparison to other previously known systems associated with soiled diaper detection.

The terms and expressions that have been employed within this specification are used as terms of description and not of limitation. There is no intention in the use of such terms and expressions of excluding any equivalents of the features shown and described or portions thereof. Instead, it is recognized that various modifications are possible within the scope of the invention claimed. At times modifications of or alternatives to certain features have been explicitly described. The descriptions of modifications or alternatives for some features should not be read to exclude other modifications or alternatives not so described, nor should the lack of description of modifications or alternatives for other features be read to exclude such.

What is claimed is:

1. A system for detecting diaper soiling, comprising:
a diaper, comprising a conductive interior; and
the detection module, comprising:
an accelerometer configured to detect the quantity of substances contained within the diaper,
a battery,
a plurality of electrodes communicatively linked to the battery configured to detect the type of substances contained within the diaper,
the battery and the plurality of electrodes configured to create a circuit when placed in contact with the conductive interior of a diaper, and
a communicative linkage.

2. The system of claim 1, the diaper further comprising a dry substrate to facilitate completion of a circuit when placed in contact with a detection module.

3. The system of claim 1, the diaper further comprising a conductive strip to facilitate completion of a circuit when placed in contact with a detection module.

4. The system of claim 2, the dry substrate consisting of a dried tomato juice solution.

5. A detection module for detecting the presence of substances within a diaper, comprising:
an accelerometer,
an inner member,
a plurality of electrodes placed on at least one external surface of the inner member,
a memory unit,
a battery,
a communicative linkage, and
a locking mechanism.

6. The detection module of claim 5, the plurality of electrodes arranged such that at least one of the plurality of electrodes and at least one other of the plurality of electrodes are placed on opposing faces of the inner member.

7. A diaper, comprising a conductive strip and a dry substrate directly in contact with the conductive strip.

8. The diaper of claim 7, the dry substrate consisting of a dried tomato juice solution.
